(19) United States
(12) Patent Application Publication
(10) Pub. No.: US 2026/0100277 A1
(43) Pub. Date: Apr. 9, 2026

(54) ULTRASONIC DIAGNOSTIC APPARATUS, ULTRASONIC DIAGNOSTIC METHOD, AND RECORDING MEDIUM

(71) Applicant: KONICA MINOLTA, INC., Tokyo (JP)

(72) Inventor: Takashi SAKAI, Yokohama-shi (JP)

(73) Assignee: KONICA MINOLTA, INC., Tokyo (JP)

(21) Appl. No.: 19/350,968

(22) Filed: Oct. 6, 2025

(30) Foreign Application Priority Data
Oct. 9, 2024 (JP) 2024-176945

(51) Int. Cl. G16H 40/67; A61B 8/00; G06F 3/14

(52) U.S. Cl. CPC G16H 40/67; A61B 8/464; A61B 8/565; G06F 3/1454

(57) ABSTRACT

An ultrasonic diagnostic apparatus includes a hardware processor that: displays an ultrasonic image on a screen; in response to a screen mirroring request from a portable terminal connected via a network, starts a session with the portable terminal for image transmission; and periodically transmits contents displayed on the screen through the session. In response to the screen mirroring request from the portable terminal, the hardware processor performs control: when a number of a previous session already started is less than a session limit number, to start the session with the portable terminal that has made the screen mirroring request; and, when the number of the previous session already started is equal to or greater than the session limit number, to acquire predetermined information, and to end the previous session already started to newly start the session with the portable terminal that has made the screen mirroring request.

The entire disclosure of Japanese Patent Application No. 2024-176945 filed on October 9, 2024, is incorporated herein by reference in its entirety.

### BACKGROUND OF THE INVENTION

### TECHNICAL FIELD

The present disclosure relates to an ultrasonic diagnostic apparatus, an ultrasonic diagnostic method, and a recording medium.

### DESCRIPTION OF RELATED ART

A screen mirroring technology is known in which an ultrasonic diagnostic apparatus and a portable terminal such as a tablet are wirelessly connected and contents being displayed on a display part of the ultrasonic diagnostic apparatus is displayed on a screen of the portable terminal in real time. JP 2017-51610A describes that an ultrasonic diagnostic apparatus transmits ultrasonic images to a terminal device without reducing the frame rate of the ultrasonic images, and the terminal device can view the ultrasonic images in real time.

### SUMMARY OF THE INVENTION

In some cases, screen mirroring is performed by simultaneously connecting a plurality of portable terminals to a single ultrasonic diagnostic apparatus. In this case, when the number of terminals connected to the ultrasonic diagnostic apparatus increases, the processing load and network load of the ultrasonic diagnostic apparatus increase. Therefore, the number of portable terminals connectable to the ultrasonic diagnostic apparatus is limited to a certain number.

In the related art, when the number of portable terminals connected to an ultrasonic diagnostic apparatus exceeds the limit, control is performed to give priority to the connection of a portable terminal that has started screen mirroring earlier and reject a new connection from another portable terminal. However, some users may leave the previously connected portable terminal(s) idle without using screen mirroring. In such a case, there is a problem that another portable terminal that is to newly use screen mirroring cannot use screen mirroring.

Therefore, in order to solve the above-described problem, an object of the present disclosure is to provide an ultrasonic diagnostic apparatus, an ultrasonic diagnostic method, and a recording medium capable of effectively utilizing screen mirroring by controlling the connection priority of portable terminals to the ultrasonic diagnostic apparatus.

To achieve at least one of the abovementioned objects, an ultrasonic diagnostic apparatus reflecting one aspect of the present invention comprises a hardware processor that: displays an ultrasonic image on a screen; in response to a screen mirroring request made by a portable terminal connected via a network, starts a session with the portable terminal for image transmission; and periodically transmits contents displayed on the screen through the session, wherein, in response to the screen mirroring request made by the portable terminal, the hardware processor performs control: when a number of a previous session that has already been started is less than a session limit number, to start the session with the portable terminal that has made the screen mirroring request; and, when the number of the previous session that has already been started is equal to or greater than the session limit number, to acquire predetermined information, and end the previous session that has already been started to newly start the session with the portable terminal that has made the screen mirroring request.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a diagram illustrating an example of a schematic configuration of an ultrasonic diagnostic system according to a first embodiment;

FIG. 2 is an example of a block diagram illustrating an ultrasonic diagnostic apparatus according to the first embodiment;

FIG. 3 is a diagram illustrating an example of a table for managing connected device information on portable terminals that have started respective sessions with the ultrasonic diagnostic apparatus according to the first embodiment;

FIG. 4 is an example of a block diagram illustrating one of the portable terminals according to the first embodiment;

FIG. 5 is a flowchart illustrating an example of an operation of the ultrasonic diagnostic apparatus when screen mirroring is performed between the ultrasonic diagnostic apparatus and the portable terminal(s) according to the first embodiment;

FIG. 6 is a flowchart illustrating an example of an operation of the ultrasonic diagnostic apparatus when screen mirroring is performed between the ultrasonic diagnostic apparatus and the portable terminal(s) according to a second embodiment;

FIG. 7 is a diagram illustrating an example of a selection screen that is superimposed on a screen of a display part of the portable terminal that has transmitted a screen mirroring request according to the second embodiment;

FIG. 8 is a diagram illustrating an example of a selection screen displayed on the display part of the portable terminal that has started a session earliest according to a modification example of the second embodiment;

FIG. 9 is a flowchart illustrating an example of an operation of the ultrasonic diagnostic apparatus when screen mirroring is performed between the ultrasonic diagnostic apparatus and the portable terminal(s) according to a third embodiment; and

FIG. 10 is a diagram illustrating an example of a selection screen that is superimposed on a screen of a display part of the ultrasonic diagnostic apparatus according to the third embodiment.

### DETAILED DESCRIPTION

Hereinafter, an ultrasonic diagnostic apparatus, an ultrasonic diagnostic method, and a recording medium according to preferred embodiments of the present disclosure will be described in detail with reference to the drawings. However, the scope of the invention is not limited to the disclosed embodiments.

### <First Embodiment>

### [Exemplary Configuration of Ultrasonic Diagnostic System 1]

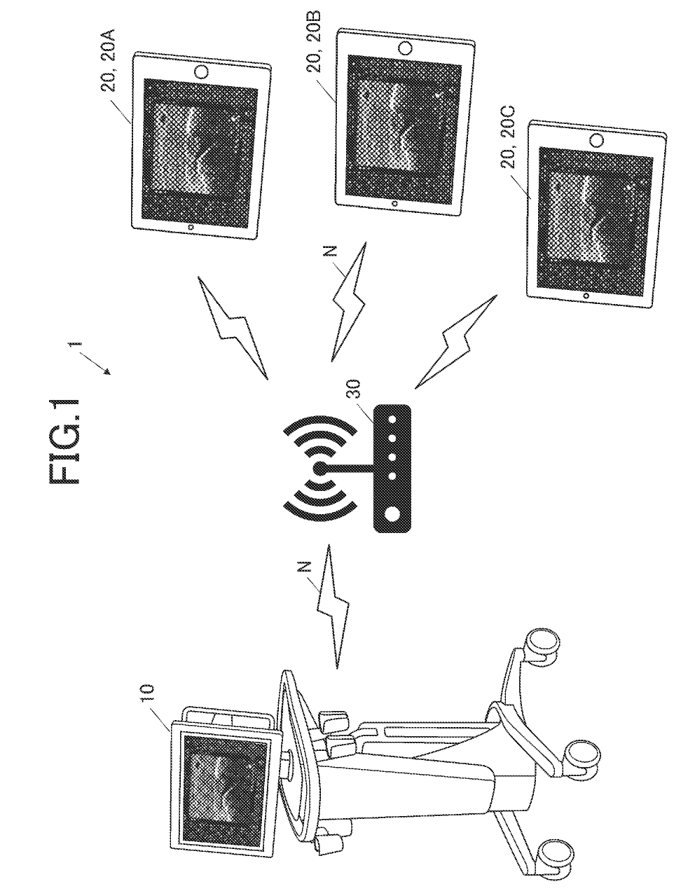

FIG. 1 is a diagram illustrating an example of a schematic configuration of an ultrasonic diagnostic system 1 according to a first embodiment. The ultrasonic diagnostic system 1 includes an ultrasonic diagnostic apparatus 10, portable terminals 20, and an access point 30. The ultrasonic diagnostic apparatus 10 and the portable terminals 20 are connected to each other by a wireless communication network N via, for example, the access point 30. The wireless communication network N includes, for example, a wireless LAN such as Wi-Fi. The wireless communication network N may be a direct connection (P2P) using short-range wireless communication such as Bluetooth without using the access point 30. Furthermore, although an example in which the ultrasonic diagnostic apparatus 10 and the portable terminals 20 are connected to each other by wireless communication has been described, the present disclosure is not limited to this. Wired communication using wiring such as a LAN cable may be used, for example.

The ultrasonic diagnostic apparatus 10 transmits ultrasonic waves to a subject, receives reflected waves reflected inside the subject, and generates tomographic images of tissues, organs, and the like of the subject based on the received reflected waves. The ultrasonic diagnostic apparatus 10 displays the generated ultrasonic images of the examination site on a screen. The ultrasonic diagnostic apparatus 10 starts a session for ultrasonic image transmission when receiving a screen mirroring request from one of the portable terminals 20 connected via the network N. During the session, contents displayed on the screen including the ultrasonic images and the like are periodically transmitted to the portable terminal 20. A session is defined as the start to the end of communication. For example, the session includes the entire sequence of communication from establishing a connection to disconnecting. The mirroring refers to transmitting contents being displayed on the screen of the ultrasonic diagnostic apparatus 10 to the portable terminal 20 and displaying the same contents as those displayed on the ultrasonic diagnostic apparatus 10 on a screen of the portable terminal 20 in real time.

Each of the portable terminals 20 is, for example, a tablet, a smartphone, or a notebook personal computer. The portable terminal 20 transmits a screen mirroring request to the ultrasonic diagnostic apparatus 10 and starts a session with the ultrasonic diagnostic apparatus 10. With the start of the session, the portable terminal 20 receives ultrasonic images and the like from the ultrasonic diagnostic apparatus 10 and displays the received ultrasonic images and the like on the screen in real time. In the present embodiment, three portable terminals of a first portable terminal 20A, a second portable terminal 20B, and a third portable terminal 20C are exemplified as the portable terminals 20 that perform respective sessions with the ultrasonic diagnostic apparatus 10, but the number of portable terminals 20 is not limited to three.

### [Exemplary Configuration of Ultrasonic Diagnostic Apparatus 10]

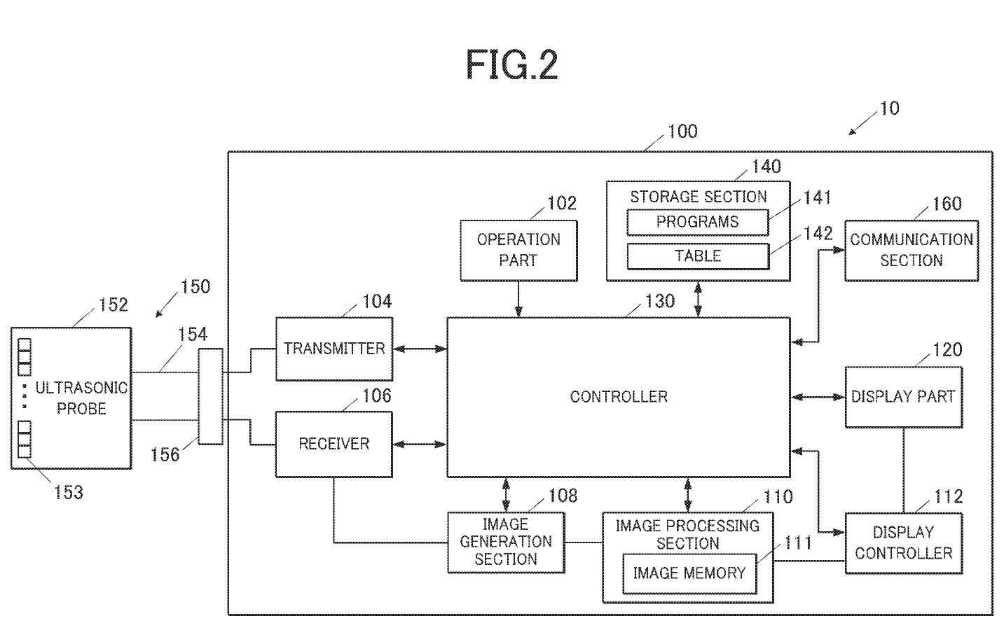

FIG. 2 is an example of a block diagram illustrating the ultrasonic diagnostic apparatus 10 according to the first embodiment. The ultrasonic diagnostic apparatus 10 includes an apparatus body 100 and an ultrasonic probe 150 connected to the apparatus body 100. The apparatus body 100 is provided with an operation part 102 and a display part 120. The apparatus body 100 includes a transmitter 104, a receiver 106, an image generation section 108, an image processing section 110, a display controller 112, a controller (hardware processor) 130, a storage section 140, and a communication section 160.

The operation part 102 includes, for example, at least one of a plurality of buttons, a trackball, a mouse, a touch screen combined with the display part 120, or the like. The operation part 102 receives input instructions by various user operations, converts the received input instructions into electrical signals, and outputs the electrical signals to the controller 130.

The transmitter 104 supplies a drive signal, which is an electrical signal, to the ultrasonic probe 150 under the control of the controller 130. The transmitter 104 includes, for example, a clock generation circuit, a delay circuit, and a pulse generation circuit. The clock generation circuit generates a clock signal for determining the transmission timing and transmission frequency of the drive signal. The delay circuit sets a delay time for each path provided in each probe 153 to be described later, and delays transmission of the drive signal by the set delay time. The delay circuit focuses a transmission beam constituted by ultrasonic waves. The pulse generation circuit generates a pulse signal as a drive signal at a predetermined cycle. For example, the transmitter 104 drives a consecutive portion of the plurality of probes 153 to generate ultrasonic waves. The transmitter 104 performs scanning while shifting the probes 153 to be driven in the azimuth direction each time the ultrasonic waves are generated.

The receiver 106 receives a reception signal, which is the electrical signal, from the ultrasonic probe 150 under the control of the controller 130. The receiver 106 includes, for example, an amplifier, an A/D conversion circuit, and a phasing addition circuit. The amplifier amplifies the reception signal at a preset amplification factor for each path provided in each probe 153. The A/D conversion circuit performs analog/digital conversion on the amplified reception signal. The phasing addition circuit gives a delay time to the A/D converted reception signal for each path provided in each probe 153 to adjust the time phase and adds these. The phasing addition circuit generates sound ray data (sound ray signal) by phasing addition. Note that the receiver 106 may include an amplifier for amplifying the reception signal.

The image generation section 108 performs envelope detection processing, logarithmic compression, and the like on the sound ray data supplied from the receiver 106. The image generation section 108 further adjusts at least one of the dynamic range or the gain of the sound ray data to convert the brightness, thereby generating B-mode image data. The B-mode image data represents the strength of the reception signals by brightness and is tomographic image information on tissues of the subject. Note that the image data is not limited to the B-mode image data in the B-mode. Examples of the other scan modes (image modes) include an A-mode, an M-mode, and a scan mode using the Doppler method. The Doppler method includes, for example, a color Doppler mode and a PWD. The B-mode is an abbreviation for Brightness mode. The A-mode is an abbreviation for Amplitude mode. The M-mode is an abbreviation for Motion mode. The PWD is an abbreviation for Pulsed Wave Doppler.

The image processing section 110 performs image processing on the B-mode image data output from the image generation section 108, for example, according to various image parameters being set. The image processing section 110 includes an image memory 111 constituted by a semiconductor memory such as a DRAM. The DRAM is an abbreviation for Dynamic Random Access Memory. Under the control of the controller 130, the image processing section 110 stores the B-mode image data that has undergone the image processing, frame by frame, in the image memory 111. Under the control of the controller 130, the image processing section 110 sequentially outputs the image data generated as described above to the display controller 112.

Under the control of the controller 130, the display controller 112 generates an image signal for display by performing coordinate conversion or the like on the received image data. The display controller 112 outputs the generated image signal for display to the display part 120.

The display part 120 displays an ultrasonic image of a tissue, an organ, and the like of the subject based on the image signal for display output from the display controller 112 on a screen under the control of the controller 130. The ultrasonic image may be a still image or a moving image. The display part 120 may be a display device connected to the apparatus body 100 via, for example, wiring such as a cable, or the network N.

The controller 130 is a computer and includes a processor such as a CPU. The CPU is an abbreviation for Central Processing Unit. The CPU can read programs 141 stored in the storage section 140, develop the programs in a memory such as a RAM, and execute processing related to an ultrasonic examination, processing related to screen mirroring, and the like in cooperation with the programs 141. In the present embodiment, in a case where a screen mirroring request is received from one of the portable terminals 20, when the number of started sessions is less than a session limit number, the controller 130 performs control to start a session with the portable terminal that has made the screen mirroring request. On the other hand, when the number of started sessions is equal to or greater than the session limit number, the controller 130 performs control to acquire predetermined information, ends one of the started sessions, and starts a new session with the portable terminal 20 that has made the screen mirroring request.

The storage section 140 includes at least one storage module, for example, an HDD, an SSD, a ROM, and a RAM. The HDD is an abbreviation for Hard Disk Drive. The SSD is an abbreviation for Solid State Drive. The ROM is an abbreviation for Read Only Memory. The RAM is an abbreviation for Random Access Memory. The storage section 140 stores, for example, system programs, application programs, and various types of data received by the communication section 160. For example, the storage section 140 stores the programs 141 for executing the processing related to an ultrasonic examination, the processing related to screen mirroring, and the like. Furthermore, the storage section 140 stores a table 142 for managing connected device information on the portable terminals 20 that have started respective sessions with the ultrasonic diagnostic apparatus 10.

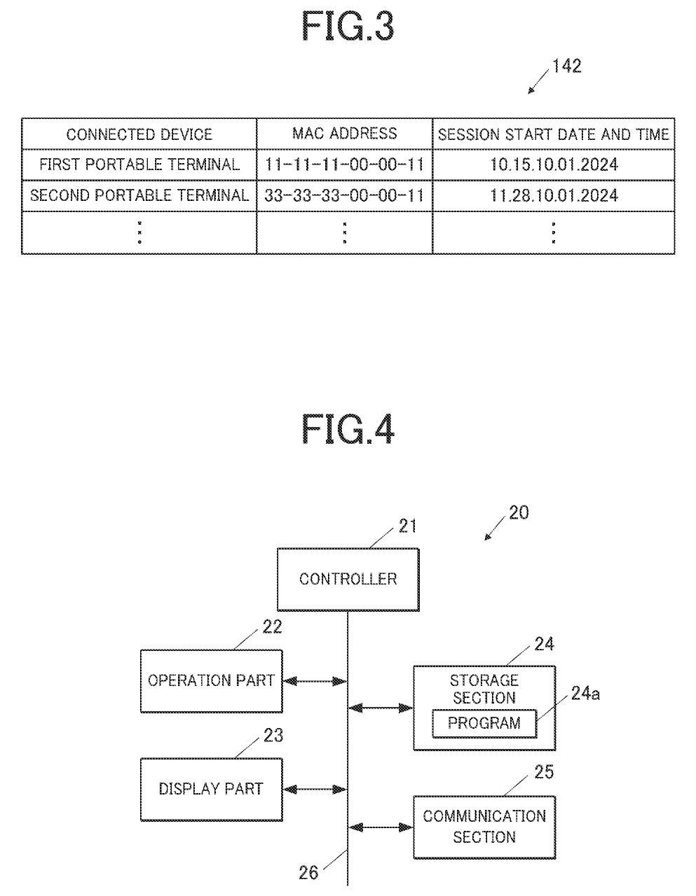

FIG. 3 illustrates an example of the table 142 for managing the connected device information on the portable terminals 20 with which the respective sessions have been started according to the first embodiment. In the table 142, an MAC address, which is identification information of the portable terminal 20 that has started a session with the ultrasonic diagnostic apparatus 10, and the date and time when the session with the ultrasonic diagnostic apparatus 10 was started are stored in association with each other. The identification information may be, for example, something other than the MAC address, such as an IP address. Furthermore, a port number may be associated in the table 142. The portable terminal 20 for which the session with the ultrasonic diagnostic apparatus 10 has been ended may be deleted from the table 142, or flag information indicating that the session has ended may be added to the connected device information corresponding to the portable terminal 20.

The communication section 160 includes, for example, an NIC, a LAN adapter, and a communication module including a receiver and a transmitter. The NIC is an abbreviation for Network Interface Card. The communication section 160 communicates various data, information, and the like with an external device such as the portable terminals 20 via the network N, for example.

The ultrasonic probe 150 includes a head part 152, a cable 154, and a connector 156. The head part 152 is a portion to be pressed against the body surface of a subject person. The head part 152 is provided with a plurality of probes 153 formed of piezoelectric elements. Each of the plurality of probes 153 transmits an ultrasonic wave to the subject based on a drive signal transmitted from the apparatus body 100 and receives a reflected wave reflected by a target tissue in the subject. For example, the plurality of probes 153 may be arranged in a scanning direction in a one-dimensional array or may be arranged in a two-dimensional array (matrix). The number of probes 153 can be suitably set. As a scanning method of the ultrasonic probe 150, a linear scanning method, a convex scanning method, a sector scanning method, or the like can be adopted.

The cable 154 has one end electrically connected to the head part 152 and the other end electrically connected to the connector 156. The connector 156 is connected to the apparatus body 100. Note that the communication between the apparatus body 100 and the ultrasonic probe 150 is not limited to wired communication using the cable 154. The communication method between the apparatus body 100 and the ultrasonic probe 150 may be wireless communication using UWB or the like. The UWB is an abbreviation for Ultra Wide Band.

### [Exemplary Configuration of Portable Terminal 20]

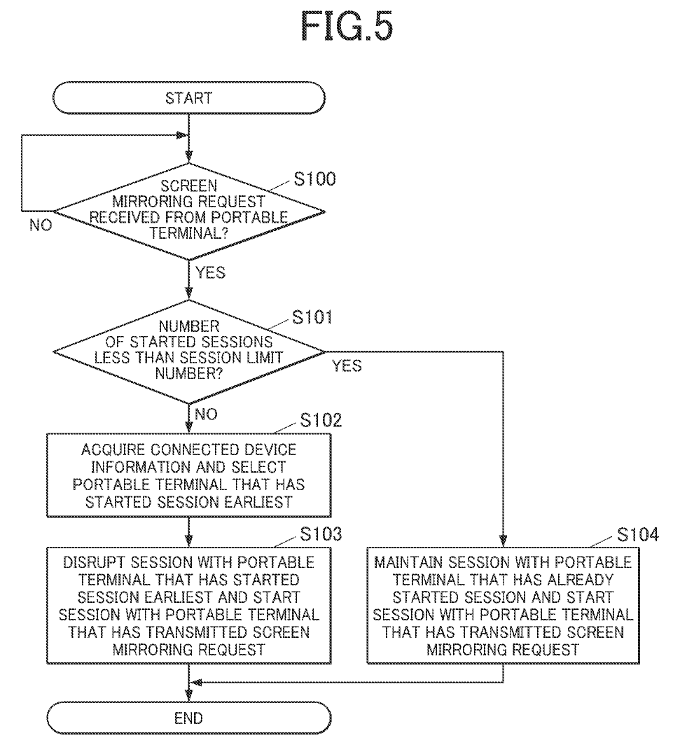

FIG. 4 illustrates an example of a block diagram of the portable terminal 20 according to the first embodiment. The portable terminal 20 includes a controller 21, an operation part 22, a display part 23, a storage section 24, and a communication section 25. The controller 21, the operation part 22, the display part 23, the storage section 24, and the communication section 25 are connected to each other via wiring such as a bus 26.

The controller 21 includes, for example, a processor such as a CPU and a GPU. The CPU is an abbreviation for Central Processing Unit. The GPU is an abbreviation for Graphics Processing Unit. The processor may be constituted by one processor or may be constituted by two or more processors. The controller 21 executes, for example, a program 24a stored in a memory, the storage section 24, or the like to implement a process of performing screen mirroring with the ultrasonic diagnostic apparatus 10.

The operation part 22 includes at least one of a touch screen, a switch, a button, a mouse, or a keyboard that is integrally combined with the display part 23. The operation part 22 receives instructions corresponding to various input operations from a user, and outputs operation signals based on the received instructions to the controller 21. For example, the operation part 22 receives a screen mirroring request or the like from the user.

The display part 23 includes, for example, a display such as a liquid crystal display or an organic EL display. The EL is an abbreviation for Electro Luminescence. The display part 23 displays a GUI for receiving various input operations from the user. The display part 23 displays the same screen contents as those on the screen of the ultrasonic diagnostic apparatus 10 when screen mirroring is performed. The GUI is an abbreviation for Graphical User Interface.

The storage section 24 includes at least one storage module, for example, an HDD, an SSD, a ROM, and a RAM. The HDD is an abbreviation for Hard Disk Drive. The SSD is an abbreviation for Solid State Drive. The ROM is an abbreviation for Read Only Memory. The storage section 24 stores, for example, various programs such as system programs and an application program, various data, and the like. For example, the storage section 24 stores the program 24a for performing screen mirroring with the ultrasonic diagnostic apparatus 10.

The communication section 25 includes, for example, a communication module including an NIC, a receiver, and a transmitter. The NIC is an abbreviation for Network Interface Card. The communication section 25 communicates various kinds of signals, and data with the ultrasonic diagnostic apparatus 10 via the network N.

### [Exemplary Operation of Ultrasonic Diagnostic Apparatus 10]

FIG. 5 is a flowchart illustrating an example of an operation of the ultrasonic diagnostic apparatus 10 when screen mirroring is performed between the ultrasonic diagnostic apparatus 10 and the portable terminal(s) 20 according to the first embodiment. The controller 130 of the ultrasonic diagnostic apparatus 10 implements processes including control steps illustrated in FIG. 5 by executing the programs 141 and the like stored in the storage section 140.

The controller 130 determines whether a screen mirroring request has been received from one of the portable terminals 20 via the communication section 160 (step S100). If the controller 130 determines that a screen mirroring request has been received from one of the portable terminals 20 via the communication section 160, the controller 130 advances the process to step S101. In the present embodiment, a case where a screen mirroring request is newly received from the third portable terminal 20C illustrated in FIG. 1 will be described. On the other hand, if the controller 130 determines that a screen mirroring request has not been received from one of the portable terminals 20 via the communication section 160, the controller 130 continues to monitor for the reception of a screen mirroring request or the like from one of the portable terminals 20 or the like.

The controller 130 determines whether the number of started sessions of the portable terminals 20 is less than a preset session limit number (step S101). In this case, the controller 130 may acquire information on the number of started sessions by counting the number of portable terminals 20 that have started the sessions with reference to the table 142. The controller 130 may include a function of a counting unit that counts the number of portable terminals 20 that have started sessions.

If the controller 130 determines that the number of started sessions of the portable terminals 20 that have started the sessions with the ultrasonic diagnostic apparatus 10 is not less than the preset session limit number, the controller 130 advances the process to step S102. That is, this is a case where the number of started sessions of the portable terminals 20 that have already started the sessions with the ultrasonic diagnostic apparatus 10 is equal to or greater than the session limit number. An example of the case where the number of started sessions is equal to or greater than the session limit includes a case where the session limit number is two and the number of started sessions is two, that is, two of the portable terminals, the first portable terminal 20A and the second portable terminal 20B, have started the sessions.

The controller 130 acquires, from the table 142 stored in the storage section 140, the connected device information on the portable terminal 20 that has started the session with the ultrasonic diagnostic apparatus 10 earliest (step S102). For example, the controller 130 refers to the table 142 and compares the date and time when the first portable terminal 20A started the session with the date and time when the second portable terminal 20B started the session. Since the date and time of the first portable terminal 20A is earlier than the date and time of the second portable terminal 20B, the controller 130 selects the first portable terminal 20A as the connected device information on the portable terminal 20 that has started the session with the ultrasonic diagnostic apparatus 10 earliest.

Based on the acquired connected device information, the controller 130 disrupts the session with the portable terminal 20 that has started the session earliest by rejecting the session and starts a session with the portable terminal 20 that has transmitted the screen mirroring request (step S103). For example, when the portable terminal 20 that has started the session earliest is the first portable terminal 20A, the controller 130 disrupts the session with the first portable terminal 20A. Furthermore, when the portable terminal 20 that has transmitted the screen mirroring request is the third portable terminal 20C, the controller 130 newly starts a session with the third portable terminal 20C.

On the other hand, if the controller 130 determines in step 101 that the number of started sessions of the portable terminals 20 that have already started the sessions with the ultrasonic diagnostic apparatus 10 is less than the preset session limit number, the controller 130 advances the process to step S104. An example of the case where the number of started sessions is less than the session limit number includes a case where the session limit number is two and the number of started sessions is one, that is, one of the portable terminals, the first portable terminal 20A, has started the session. In this case, one connection slot is available for one of the portable terminals 20 that can newly start respective sessions.

In this case, the controller 130 starts a new session with the portable terminal 20 that has transmitted the screen mirroring request, while maintaining the session with the portable terminal 20 that has already started the session (step S104). For example, when a session has been started between the ultrasonic diagnostic apparatus 10 and the first portable terminal 20A, the controller 130 maintains the session with the first portable terminal 20A. If the portable terminal that has transmitted the screen mirroring request is the third portable terminal 20C, the controller 130 newly starts a session with the third portable terminal 20C. That is, the ultrasonic diagnostic apparatus 10 performs screen mirroring with two of the portable terminals, the first portable terminals 20A and the third portable terminals 20C.

According to the first embodiment, when a screen mirroring request is received from one of the portable terminals 20 and the number of started sessions is equal to or greater than the session limit number, the controller 130 disrupts the session with the portable terminal 20 that has already started the session. Further, when the above conditions are satisfied, the controller 130 starts a new session with the portable terminal 20 that has made the screen mirroring request. That is, in the first embodiment, priority is given to the session with the portable terminal 20 that has transmitted the screen mirroring request later. This allows a session of a portable terminal 20 that requires screen mirroring to take priority over a session of a portable terminal 20 that has started the session earlier and is left idle without using screen mirroring. As a result, even when the session limit number is set for the purpose of reducing the processing load or the like, the screen mirroring can be effectively used.

### <Second Embodiment>

In a second embodiment, when the number of started sessions of the portable terminals 20 is equal to or greater than the session limit number, the portable terminal 20 can select whether to prioritize its own connection. Hereinafter, the differences from the first embodiment will be mainly described, constituent elements substantially common to the first embodiment will be assigned with the same reference signs, and common description will be omitted or simplified.

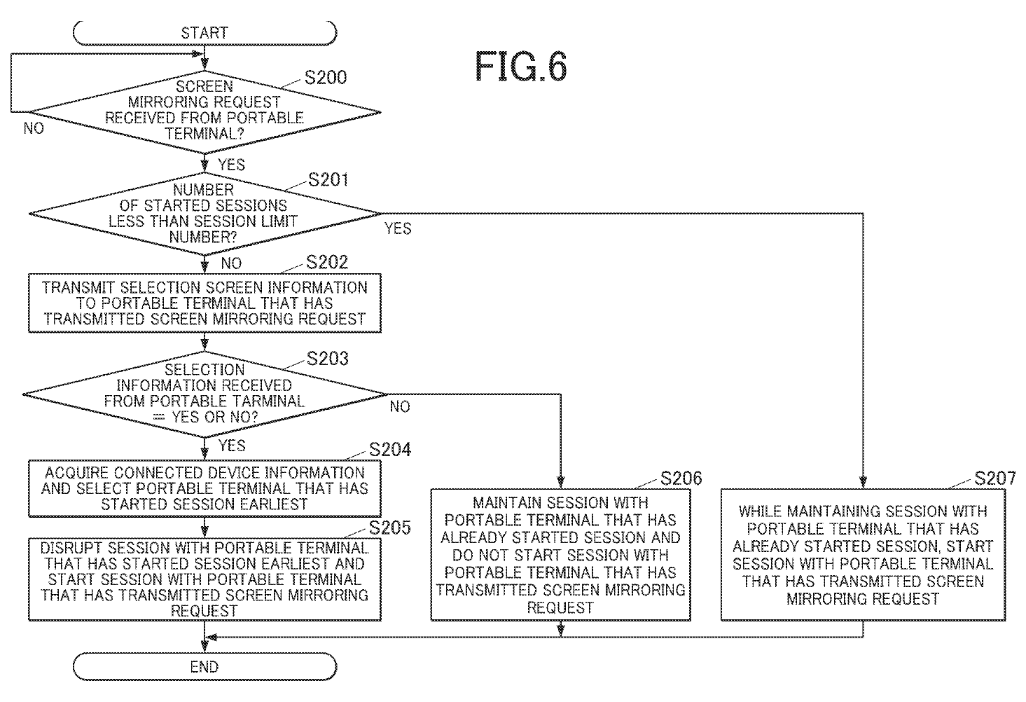

FIG. 6 is a flowchart illustrating an example of an operation of the ultrasonic diagnostic apparatus 10 when screen mirroring is performed between the ultrasonic diagnostic apparatus 10 and the portable terminal(s) 20 according to the second embodiment. The controller 130 of the ultrasonic diagnostic apparatus 10 implements processes including control steps illustrated in FIG. 6 by executing the programs 141 and the like stored in the storage section 140.

The controller 130 determines whether a screen mirroring request has been received from one of the portable terminals 20 via the communication section 160 (step S200). If the controller 130 determines that a screen mirroring request has been received from one of the portable terminals 20 via the communication section 160, the controller 130 advances the process to step S201. In the present embodiment, a case where a screen mirroring request is newly received from the third portable terminal 20C illustrated in FIG. 1 will be described. On the other hand, if the controller 130 determines that a screen mirroring request has not been received from one of the portable terminals 20 via the communication section 160, the controller 130 continues to monitor for the reception of a screen mirroring request or the like from one of the portable terminals 20 or the like.

The controller 130 determines whether the number of started sessions of the portable terminals 20 that have started the sessions with the ultrasonic diagnostic apparatus 10 is less than a preset session limit number (step S201). In this case, the controller 130 may acquire information on the number of started sessions by counting the number of portable terminals 20 that have started the sessions with reference to the table 142.

If the controller 130 determines that the number of started sessions of the portable terminals 20 is not less than the preset session limit number, the controller 130 advances the process to step S202. That is, this is a case where the number of started sessions of the portable terminals 20 that have already started the sessions with the ultrasonic diagnostic apparatus 10 is equal to or greater than the session limit number. An example of the case where the number of started sessions is equal to or greater than the session limit includes a case where the session limit number is two and the number of started sessions is two, that is, two of the portable terminals, the first portable terminal 20A and the second portable terminal 20B, have started the sessions.

The controller 130 transmits selection screen information via the communication section 160 to the portable terminal 20 that has transmitted the screen mirroring request (step S202). The selection screen information is a popup window that is superimposed on a screen of the display part 23 of the portable terminal 20 that has transmitted the screen mirroring request. The selection screen information includes image information for selecting whether to start a session with the ultrasonic diagnostic apparatus 10. Upon receiving the selection screen information from the ultrasonic diagnostic apparatus 10 via the communication section 25, the controller 21 of the portable terminal 20 superimposes a selection screen 200 based on the received selection screen information on the screen of the display part 23.

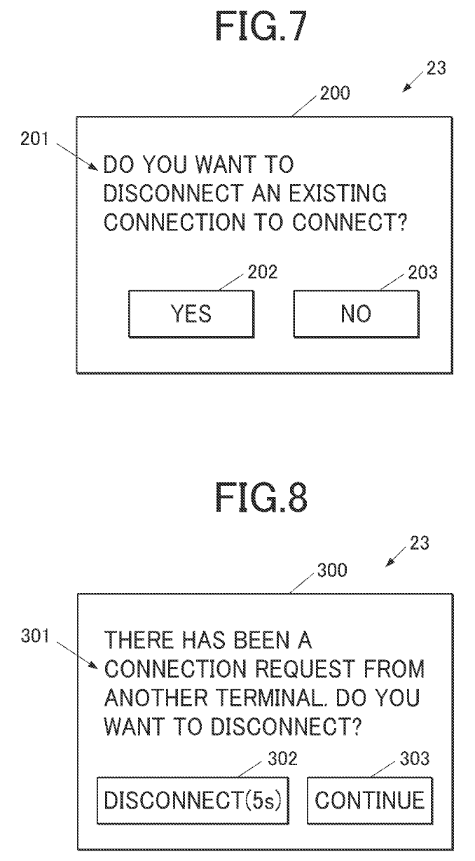

FIG. 7 is a diagram illustrating an example of the selection screen 200 that is superimposed on the screen of the display part 23 of the portable terminal 20 that has transmitted the screen mirroring request according to the second embodiment. On the selection screen 200, text information 201, for example, “DO YOU WANT TO DISCONNECT AN EXISTING CONNECTION TO CONNECT?” is displayed. Furthermore, a YES button 202 and a NO button 203 are displayed on the selection screen 200. The YES button 202 is a button for receiving an instruction to disconnect the connection of the first portable terminal 20A that has started the session earlier to start a session with priority given to the connection of the third portable terminal 20C itself. The NO button 203 is a button for receiving an instruction to maintain the connection of the first portable terminal 20A that has started the session earlier and not to connect the third portable terminal 20C itself.

The controller 130 determines whether selection information related to an operation on the selection screen 200 has been received from the portable terminal 20 that has transmitted the selection screen information (step S203). The selection information includes either operation information corresponding to the YES button 202 or operation information corresponding to the NO button 203 selected on the selection screen 200 of the portable terminal 20. When the received selection information includes the operation information corresponding to the YES button 202, the controller 130 advances the process to step S204.

The controller 130 acquires, from the table 142 stored in the storage section 140, the connected device information on the portable terminal 20 that has started the session with the ultrasonic diagnostic apparatus 10 earliest (step S204). For example, the controller 130 refers to the table 142 and compares the date and time when the first portable terminal 20A started the session with the date and time when the second portable terminal 20B started the session. Since the date and time of the first portable terminal 20A is earlier than the date and time of the second portable terminal 20B, the controller 130 selects the first portable terminal 20A as the connected device information on the portable terminal 20 that has started the session with the ultrasonic diagnostic apparatus 10 earliest.

Based on the acquired connected device information, the controller 130 disrupts the session with the portable terminal 20 that has started the session earliest by rejecting the session and starts a session with the portable terminal 20 that has transmitted the screen mirroring request (step S205). For example, when the portable terminal 20 that has started the session earliest is the first portable terminal 20A, the controller 130 disrupts the session with the first portable terminal 20A. When the portable terminal 20 that has transmitted the screen mirroring request is the third portable terminal 20C, the controller 130 newly starts a session with the third portable terminal 20C.

On the other hand, in step S203, when the received selection information includes the operation information corresponding to the NO button 203, the controller 130 advances the process to step S206. In this case, the controller 130 maintains the session with the portable terminal 20 that has already started the session and does not start a new session with the portable terminal 20 that has transmitted the screen mirroring request (step S206). For example, when sessions have been started between the ultrasonic diagnostic apparatus 10 and the first portable terminal 20A and between the ultrasonic diagnostic apparatus 10 and the second portable terminal 20B, the controller 130 maintains the sessions with the first portable terminal 20A and the second portable terminal 20B. When the portable terminal 20 that has transmitted the screen mirroring request is the third portable terminal 20C, the controller 130 performs control not to start a session with the third portable terminal 20C.

In addition, in step S201 above, when the controller 130 determines that the number of started sessions of the portable terminals 20 is less than the preset session limit number, the controller 130 advances the process to step S207. An example of the case where the number of started sessions is less than the session limit number includes a case where the session limit number is two and the number of started sessions is one, that is, one of the portable terminals, the first portable terminal 20A, has started the session.

Thus, the controller 130 starts a new session with the portable terminal 20 that has transmitted the screen mirroring request, while maintaining the session with the portable terminal 20 that has already started the session (step S207). For example, when a session has been started between the ultrasonic diagnostic apparatus 10 and the first portable terminal 20A, the controller 130 maintains the session with the first portable terminal 20A. Furthermore, when the portable terminal 20 that has transmitted the screen mirroring request is the third portable terminal 20C, the controller 130 newly starts a session with the third portable terminal 20C.

According to the second embodiment, when the number of started sessions is equal to or greater than the session limit number, the controller 130 causes the display part 23 of the portable terminal 20 that has transmitted the screen mirroring request to display the selection screen 200. This allows a user of the portable terminal 20 that has transmitted the screen mirroring request to select the portable terminal 20 that is to have a session with the ultrasonic diagnostic apparatus 10. Therefore, for example, when the priority of using one of the portable terminals 20 is high at the time of ultrasonic diagnosis or the like, a session of the portable terminal 20 that has started the session earlier can be disrupted, and the start of a session of the user’s own portable terminal 20 can be prioritized.

### <Modification Example of Second Embodiment>

In the second embodiment above, when the number of started sessions is greater than the session limit number, the portable terminal 20 that has transmitted the screen mirroring request can select whether to start a session, but the present disclosure is not limited to this. For example, the portable terminal 20 that has started the session earlier may be allowed to select whether to prioritize its own session.

Specifically, when a screen mirroring request is received from one of the portable terminals 20 and the number of started sessions is equal to or greater than the session limit number, the controller 130 identifies the portable terminal 20 that has started the session earliest. The controller 130 transmits selection screen information to the identified portable terminal 20 via the communication section 160. Upon receiving the selection screen information from the ultrasonic diagnostic apparatus 10, the portable terminal 20 that has started the session earliest causes the display part 23 to display a selection screen 300 based on the selection screen information.

FIG. 8 is a diagram illustrating an example of the selection screen 300 displayed on the display part 23 of the portable terminal 20 that has started the session earliest according to a modification example of the second embodiment. On the selection screen 300, text information 301, a DISCONNECT button 302, and a CONTINUE button 303 are displayed. The text information 301 is, for example, a message, “THERE HAS BEEN A CONNECTION REQUEST FROM ANOTHER TERMINAL. DO YOU WANT TO DISCONNECT?" The DISCONNECT button 302 is a button for receiving an instruction to disconnect the connection of the portable terminal 20 that has started the session earliest to start a session with priority given to the connection of the portable terminal 20 that has newly requested mirroring. In the present embodiment, when the DISCONNECT button 302 is selected, the session is disconnected after 5 seconds, for example. The CONTINUE button 303 is a button for receiving an instruction to maintain the connection of the portable terminal 20 that has started the session earlier and not to connect the portable terminal 20 that has newly requested mirroring.

When operation information corresponding to the DISCONNECT button 302 on the selection screen 300 is received from the portable terminal 20, the controller 130 performs control to disrupt the session with the portable terminal 20 that has started the session earliest and start a session with the portable terminal 20 that has transmitted the screen mirroring request. On the other hand, when operation information corresponding to the CONTNIUE button 303 on the selection screen 300 is received from the portable terminal 20, the controller 130 performs control to maintain the session with the portable terminal 20 that has started the session earlier and not to start a session with the portable terminal 20 that has transmitted the screen mirroring request.

According to the modification example of the second embodiment, when the number of started sessions is equal to or greater than the session limit number, the controller 130 causes the display part 23 of the portable terminal 20 that has started the session earlier to display the selection screen 300. This allows a user of the portable terminal 20 that has started the session earlier to select the portable terminal 20 that is to have a session with the ultrasonic diagnostic apparatus 10. Therefore, when the user is not currently using screen mirroring, the user can select to discontinue the session with the ultrasonic diagnostic apparatus 10 and give up the connection slot to another portable terminal 20.

### <Third Embodiment>

In a third embodiment, when the number of started sessions of the portable terminals 20 is equal to or greater than the session limit number, the ultrasonic diagnostic apparatus 10 can select whether to start a session with the portable terminal 20 that has made a screen mirroring request. Hereinafter, the differences from the first embodiment and the second embodiment will be mainly described, constituent elements substantially common to the first embodiment will be assigned with the same reference signs, and common description will be omitted or simplified.

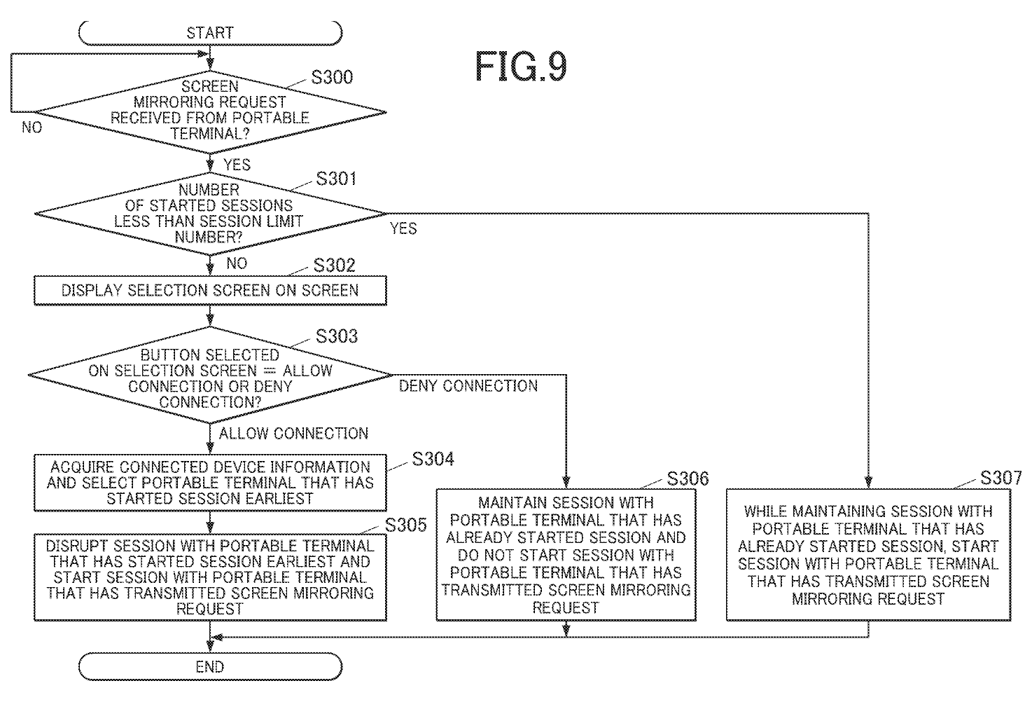

FIG. 9 is a flowchart illustrating an example of an operation of the ultrasonic diagnostic apparatus 10 when screen mirroring is performed between the ultrasonic diagnostic apparatus 10 and the portable terminal(s) 20 according to the third embodiment. The controller 130 of the ultrasonic diagnostic apparatus 10 implements processes including control steps illustrated in FIG. 9 by executing the programs 141 stored in the storage section 140.

The controller 130 determines whether a screen mirroring request has been received from one of the portable terminals 20 via the communication section 160 (step S300). If the controller 130 determines that a screen mirroring request has been received from one of the portable terminals 20 via the communication section 160, the controller 130 advances the process to step S301. On the other hand, if the controller 130 determines that a screen mirroring request has not been received from one of the portable terminals 20 via the communication section 160, the controller 130 continues to monitor for the reception of a screen mirroring request or the like from one of the portable terminals 20 or the like.

The controller 130 determines whether the number of started sessions of the portable terminals 20 is less than a preset session limit number (step S301). If the controller 130 determines that the number of started sessions is not less than the session limit number, that is, the number of started sessions is equal to or greater than the session limit number, the controller 130 advances the process to step S302.

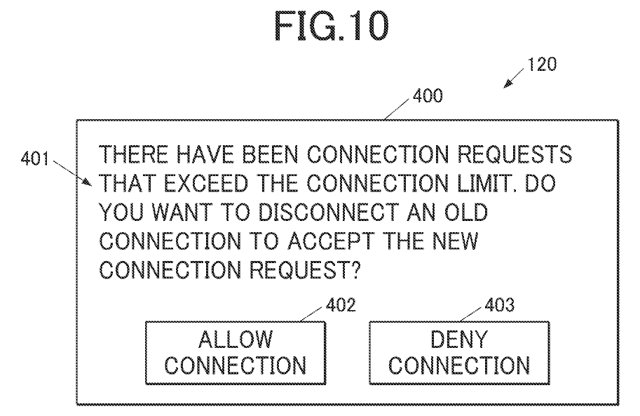

The controller 130 displays a selection screen 400 on the screen of the display part 120 for selecting whether to start a session with the portable terminal 20 that has transmitted the screen mirroring request or the like. (step S302). FIG. 10 is a diagram illustrating an example of the selection screen 400 that is superimposed on the screen of the display part 120 of the ultrasonic diagnostic apparatus 10 according to the third embodiment. On the selection screen 400, text information 401, an ALLOW CONNECTION button 402, and a DENY CONNECTION button 403 are displayed. The text information 401 is, for example, a message "THERE HAVE BEEN CONNECTION REQUESTS THAT EXCEED THE CONNECTION LIMIT. DO YOU WANT TO DISCONNECT AN OLD CONNECTION TO ACCEPT THE NEW CONNECTION REQUEST?" The ALLOW CONNECTION button 402 is a button for receiving an instruction to disconnect its own connection with the portable terminal 20 that has started the session earliest to allow a new connection with another portable terminal 20. The DENY CONNECTION button 403 is a button for receiving an instruction to maintain its own connection with the portable terminal 20 that has started the session earliest and to deny a new connection with another portable terminal 20.

The controller 130 determines whether the ALLOW CONNECTION button 402 has been selected or the DENY CONNECTION button 403 has been selected on the selection screen 400 (step S303). If the ALLOW CONNECTION button 402 has been selected on the selection screen 400, that is, if the controller 130 acquires an operation signal corresponding to the selection of the ALLOW CONNECTION button 402, the controller 130 advances the process to step S304.

The controller 130 acquires, from the table 142 stored in the storage section 140, the connected device information on the portable terminal 20 that has started the session with the ultrasonic diagnostic apparatus 10 earliest (step S304). Based on the acquired connected device information, the controller 130 disrupts the session with the portable terminal 20 that has started the session earliest by rejecting the session and starts a session with the portable terminal 20 that has transmitted the screen mirroring request (step S305).

On the other hand, in step S303, if the DENY CONNECTION button 403 has been selected on the selection screen 400, that is, if the controller 130 acquires an operation signal corresponding to the selection of the DENY CONNECTION button 403, the controller 130 advances the process to step S306. In this case, the controller 130 performs control to maintain the session with the portable terminal 20 that has already started the session and not to start a new session with the portable terminal 20 that has transmitted the screen mirroring request (step S306).

In step S301 above, if the controller 130 determines that the number of started sessions is less than the session limit number, the controller 130 advances the process to step S307. In this case, the controller 130 starts a new session with the portable terminal 20 that has transmitted the screen mirroring request, while maintaining the session with the portable terminal 20 that has already started the session (step S307).

According to the third embodiment, when the number of started sessions is equal to or greater than the session limit number, the controller 130 of the ultrasonic diagnostic apparatus 10 displays the selection screen 400 on its own display part 120. This allows a user of the ultrasonic diagnostic apparatus 10 to select the portable terminal 20 that is to have a session with the ultrasonic diagnostic apparatus 10. Therefore, for example, after checking the screen mirroring usage status of each portable terminal 20 or based on a preset connection rule or the like, the user can select the portable terminal 20 that requires screen mirroring.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

In the above embodiments, when the number of started sessions is equal to or greater than the session limit number, the session of the portable terminal 20 that has started the session with the ultrasonic diagnostic apparatus 10 earliest is disconnected, but the present disclosure is not limited to this. For example, the controller 130 or the like of the ultrasonic diagnostic apparatus 10 may acquire information on the usage status of each portable terminal 20, the browsing status of each session, and the like, and select the portable terminal 20 whose session is to be disconnected based on the acquired information.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus comprising a hardware processor that:
displays an ultrasonic image on a screen;
in response to a screen mirroring request made by a portable terminal connected via a network, starts a session with the portable terminal for image transmission; and
periodically transmits contents displayed on the screen through the session, wherein,
in response to the screen mirroring request made by the portable terminal, the hardware processor performs control:
when a number of a previous session that has already been started is less than a session limit number, to start the session with the portable terminal that has made the screen mirroring request; and,
when the number of the previous session that has already been started is equal to or greater than the session limit number,
to acquire predetermined information, and
to end the previous session that has already been started to newly start the session with the portable terminal that has made the screen mirroring request.

2. The ultrasonic diagnostic apparatus according to claim 1, wherein, when the number of the previous session that has already been started is equal to or greater than the session limit number, the hardware processor
acquires information on at least one of a previous portable terminal that has already been connected via the previous session, and
ends at least one of the previous session that has already been started with the previous portable terminal that has already been connected to newly start the session with the portable terminal that has made the screen mirroring request.

3. The ultrasonic diagnostic apparatus according to claim 1, wherein, when the number of the previous session that has already been started is equal to or greater than the session limit number, the hardware processor performs control to output, to the portable terminal that has made the screen mirroring request, selection screen information that allows a selection to be made on the portable terminal that has made the screen mirroring request, the selection being whether to end the previous session that has already been started to newly start the session with the portable terminal that has made the screen mirroring request or to withdraw the screen mirroring request.

4. The ultrasonic diagnostic apparatus according to claim 1, wherein, when the number of the previous session that has already been started is equal to or greater than the session limit number, the hardware processor allows a selection as to whether to end the previous session that has already been started to newly start the session with the portable terminal that has made the screen mirroring request or to reject the screen mirroring request.

5. The ultrasonic diagnostic apparatus according to claim 1, wherein when the number of the previous session that has already been started is equal to or greater than the session limit number, the hardware processor ends a session that has been started earliest among the previous session that has already been started.

6. An ultrasonic diagnostic method comprising:
displaying an ultrasonic image on a screen;
in response to a screen mirroring request made by a portable terminal connected via a network, starting a session with the portable terminal for image transmission; and
periodically transmitting contents displayed on the screen through the session, wherein,
in response to the screen mirroring request made by the portable terminal, the method further comprising controlling:
when a number of a previous session that has already been started is less than a session limit number, to start the session with the portable terminal that has made the screen mirroring request; and,
when the number of the previous session that has already been started is equal to or greater than the session limit number,
to acquire predetermined information, and
to end the previous session that has already been started to newly start the session with the portable terminal that has made the screen mirroring request.

7. A non-transitory computer-readable recording medium storing a program executable by a computer, the program causing a computer of an ultrasonic diagnostic apparatus that includes a hardware processor that:
displays an ultrasonic image on a screen;
in response to a screen mirroring request made by a portable terminal connected via a network, starts a session with the portable terminal for image transmission; and
periodically transmits contents displayed on the screen through the session,
in response to the screen mirroring request made by the portable terminal, to perform control:
when a number of a previous session that has already been started is less than a session limit number, to start the session with the portable terminal that has made the screen mirroring request; and,
when the number of the previous session that has already been started is equal to or greater than the session limit number,
to acquire predetermined information, and
to end the previous session that has already been started to newly start the session with the portable terminal that has made the screen mirroring request.
